(19) United States
(12) Patent Application Publication
(10) Pub. No.: US 2026/0103755 A1
(43) Pub. Date: Apr. 16, 2026

(54) USE OF MICRORNAS TO DIAGNOSE CANINE HEARTWORM INFECTION

(71) Applicant: MI:RNA Ltd., Edinburgh (GB)

(72) Inventors: Eve Hanks, Edinburgh (GB); Robert Coultous, Whitburn (GB)

(21) Appl. No.: 19/272,934

(22) Filed: Jul. 17, 2025

Related U.S. Application Data
(60) Provisional application No. 63/672,561, filed on Jul. 17, 2024.

(51) Int. Cl. C12Q 1/6883; G16B 25/10; G16B 40/20; G16H 50/20

(52) U.S. Cl. CPC C12Q 1/6883; G16B 25/10; G16B 40/20; G16H 50/20; C12Q 2600/158; C12Q 2600/166; C12Q 2600/178; G01N 2800/26

(57) ABSTRACT

A method of assessing canine expression profiles of miRNA markers and using predictive classification algorithms to diagnose heartworm infection. The analysis of the expression level of specific miRNAs enables early detection of the heartworm infection, including low-burden and male-only infections, which are often missed by antigen tests.

### REFERENCE TO RELATED APPLICATIONS

This application claims benefit to and priority to U.S. Application Ser. No. 63/672,561, filed on Jul. 17, 2024, which is hereby incorporated by this reference in its entirety.

### SEQUENCE LISTING

The application contains a Sequence Listing which has been submitted electronically in .XML format and is hereby incorporated by reference in its entirety. Said .XML copy, created on Nov. 6, 2025, is named “068075.012US.xml” and is 73,056 bytes in size. The sequence listing contained in this .XML file is part of the specification and is hereby incorporated by reference herein in its entirety.

### FIELD

The present invention relates to isolated nucleic acid molecules known as microRNAs (miRNAs) and miRNA precursor molecules and their use in diagnosis and therapy. The invention also relates to a method and a kit for diagnosing heartworm infection and disease in a subject. In one embodiment the subject is contemplated to be a dog, but infection in other hosts, such as cats and humans, is contemplated.

### BACKGROUND

Heartworm disease, or dirofilariasis, is a serious and potentially fatal condition in dogs caused by the filarial nematode Dirofilaria immitis. Infective larvae, transmitted into the host via mosquito bites, migrate through the tissues and eventually reach the pulmonary arteries and heart. Transmission through mosquitoes leads to a higher prevalence of canine heartworm in southern regions of the United States and Europe. While some (up to 10%) infected animals may remain asymptomatic, the presence of adult heartworms leads to severe pulmonary hypertension, right-sided heart failure, and potentially death if untreated (Ames M. K., Vet. Parasitol., 2020). In the early stages of disease, a dog typically shows little to no symptoms, allowing the disease to progress in severity.

To identify heartworm disease before the development of symptoms, dogs can be tested at routine visits. Currently, the diagnosis of heartworm infection in dogs mainly relies on the detection of microfilaria antigens derived from the reproductive tract of adult female worms in the blood. Although this test is widely used due to its high sensitivity and specificity, it has limitations in detecting low-burden infections and early stages of infection, where adult worms are not present (American Heartworm Society Quarterly Update Autumn 2016). Moreover, imaging techniques such as radiography and echocardiography can also provide visual confirmation of infective larvae. However, these are often used as secondary diagnostic tools due to their cost and the need for specialized equipment and expertise (McCall J. W., Adv Parasitol., 2008). Treatment is complex, costly, and risky, and involves medications including melarsomine and strict reduction of activity. According to Noack et al. effective prevention and treatment is also essential to delay development of drug-resistant D. immitis populations (Noack S, et al. Int J Parasitol Drugs Drug Resist. 16:65-89 (2021)). Further as noted by Hattendorf and Luhken, incidental infections have been reported in other mammals including humans and cats therefore making a diagnostic assay for these species of interest (Hattendorf C, Luhken R, Infect Dis Poverty 14:48 (2025)). This invention seeks to provide a miRNA-based assay to improve earlier detection and predict treatment response.

### SUMMARY

In accordance with the purpose(s) of this invention, as embodied and broadly described herein, this invention, in one aspect, relates to a method for diagnosing heartworm infection in a subject, comprising the steps of: a) obtaining a sample from the subject; b) isolating miRNA molecules within a sample from a subject; c) amplifying the cDNA molecules to a detectable concentration; d) probing for the cDNA molecules complementary to the desired miRNA markers; e) determining a level of expression of the miRNA molecules within a sample from a subject by the level of cDNA molecules probed for the desired miRNA markers; and f) using one or more Artificial Intelligence (AI) model to predict the disease condition of the subject; wherein the one or more AI model compares the level of expression of each cDNA molecule with at least one pre-determined reference level cDNA molecule characteristic of a non-diseased subject wherein a deviation of the level of expression of said cDNA molecule in comparison with the at least one reference level cDNA molecule allows for the diagnosis and/or prognosis of heartworm disease, and wherein the miRNA molecules comprise a panel of reference miRNA molecules selected from miRNA molecules having at least one miRNA selected from a group consisting of nucleic acid sequence having at least 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, 41, or combinations thereof, the miRNA molecules having a reverse complement cDNA with at least 99% sequence identity to SEQ ID NO: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 or combinations thereof.

In one embodiment, the method further comprises the step of using a machine learning algorithm for predictive modelling. In another embodiment, the cDNA molecule may also be a reverse complement cDNA.

One embodiment provides a panel of miRNA molecules comprising at least six reference miRNA molecules, wherein the at least six miRNA molecules are mir20a, mir223a, mir375, mir29a, mir423a, and mir71, having at least 99% sequence identity to SEQ ID NO: 19, 21, 29, 24, 30, and 35, respectively, the miRNA molecules having a reverse complement cDNA with at least 99% sequence identity to SEQ ID NO: 60, 62, 70, 62, 71, and 76, respectively. Another embodiment provides, a panel of miRNA molecules comprising at least five reference miRNA molecules, wherein the at least five miRNA molecules are mir20a, mir223a, mir375, mir29a, and mir423a, having at least 99% sequence identity to SEQ ID NO:19, 21, 29, 24, and 30, respectively, the miRNA molecules having a reverse complement cDNA with at least 99% sequence identity to SEQ ID NO: 60, 62, 70, 62, and 71, respectively. Yet another embodiment provides, a panel of miRNA molecules comprising at least four reference miRNA molecules, wherein the at least four miRNA molecules are mir20a, mir223a, mir375, and mir71, having at least 99% sequence identity to SEQ ID NO:19, 21, 29, and 35, respectively, the miRNA molecules having a reverse complement cDNA with at least 99% sequence identity to SEQ ID NO: 60, 62, 70, and 76, respectively. Another embodiment provides, a panel of miRNA molecules comprising at least three reference miRNA molecules, wherein the at least three miRNA molecules are mir20a, mir223a, and mir375, having at least 99% sequence identity to SEQ ID NO:19, 21, and 29, respectively, the miRNA molecules having a reverse complement cDNA with at least 99% sequence identity to SEQ ID NO: 60, 62, and 70, respectively. Another embodiment provides, a panel of miRNA molecules comprising at least two reference miRNA molecules, wherein the at least two miRNA molecules are mir223 and mir71, having at least 99% sequence identity to SEQ ID NO:21 and 35, the miRNA molecules having a reverse complement cDNA with at least 99% sequence identity to SEQ ID NO: 62 and 76. One other embodiment provides a panel of miRNA molecules comprising at least one reference miRNA molecule, wherein the at least one miRNA molecule is mir223, having at least 99% sequence identity to SEQ ID NO:21, the miRNA molecules having a reverse complement cDNA with at least 99% sequence identity to SEQ ID NO:62.

In another embodiment, the method further comprises the use of a normalizer and/or control miRNA molecule, wherein the normalizer miRNA molecule is mir490, having at least 99% sequence identity to SEQ ID NO:32, the miRNA molecules having a reverse complement cDNA with at least 99% sequence identity to SEQ ID NO:73, wherein the normalizer or control miRNA molecule is an off-species control miRNA molecule. In the embodiments provided herein, the miRNAs are cell-free miRNAs.

Yet another embodiment provides that a sample is obtained from a subject, wherein the subject is a mammal, and the mammal is a dog, cat, or horse. The sample is selected from a group consisting of tissue or organ samples, blood samples, urine, saliva, milk, and cerebrospinal fluid samples, wherein the blood sample is selected from the group consisting of serum, plasma, cell-free blood, whole blood, and its components, blood-derived products, or preparations thereof.

Another aspect of the invention relates to a kit for use in performing the methods described herein, comprising means for determining the level of expression of miRNA molecules selected from a miRNA panel having one or more miRNA molecules selected from miRNA molecules having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, 41, or combinations thereof, the miRNA molecules having a reverse complement cDNA with at least 99% sequence identity to SEQ ID NO: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or combinations thereof.

One other aspect of the invention relates to a method of selecting a panel for use in heartworm diagnosis comprising the steps of: a) selecting a group of miRNA molecules or the reverse complement cDNA according to claim 1, the differential expression of which may be associated with a disease condition; b) predicting the disease condition based on a deviation of the level of expression of said miRNA molecules from steps (a); and c) reducing the number of miRNAs in the panel to a minimum number to provide a panel of miRNAs that still produces a result.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIG. 1A shows a violin plot with different miRNA expression levels.

FIG. 1B shows a generalized linear model (GLM) for miRNA samples mir20a, mir223, and mir375.

FIG. 1C shows a GLM for the same data with a subset probed for parasite-specific miRNA and the addition of mir71 to the GLM.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The terms “embodiment,” “an embodiment,” “one embodiment,” “in various embodiments,” “certain embodiments,” “some embodiments,” “other embodiments,” “certain other embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with any other embodiment whether or not explicitly described.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

A “coding sequence” or “coding region” refers to a nucleic acid molecule having sequence information necessary to produce a gene product, when the sequence is expressed.

A “probe” is defined as a nucleic acid capable of binding to a target nucleic acid of complementary sequence through one or more types of chemical bonds, usually through complementary base pairing, usually through hydrogen bond formation. A probe may include natural (i.e., A, G, C, T or U) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “microRNA” or “miRNA” or “miR” designates a non-coding RNA molecule having a length of about 17 to 25 nucleotides, specifically having a length of 17, 18, 19, 20, 21, 22, 23, 24 or 25 nucleotides which hybridizes to and regulates the expression of a coding messenger RNA.

The term “miRNA molecule” refers to any nucleic acid molecule representing the miRNA, including natural miRNA molecules, i.e. the mature miRNA, pre-miRNA, pri-miRNA.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “sample” generally refers to tissue or organ sample, blood, cell-free blood such as serum and plasma, urine, saliva, milk and cerebrospinal fluid sample.

As used herein, the term “blood sample” refers to serum, plasma, cell-free blood, whole blood and its components, blood derived products or preparations. Plasma and serum are very useful as shown in the examples.

The term “quantifying” or “quantification” as used herein refers to absolute quantification, i.e. determining the amount of the respective miRNA but also encompasses measuring the level of the respective miRNA and comparing said level with reference or control miRNA, or comparative expression to other quantified miRNA. Quantification of the respective miRNA as listed in the tables herein allow expression profiling of samples and thus allow identification of signatures associated with diseased samples, as well as identification of signatures associated with prognosis and response to treatment. The quantity of miRNAs or difference in miRNA levels can be determined by any of the methods described herein.

A “control”, “control sample”, or “reference value” or “reference level” are terms which can be used interchangeably herein, and are to be understood as a sample or standard used for comparison with the experimental sample. The control may include a sample obtained from a healthy or non-diseased subject or a subject, which is not at risk of or suffering from heartworm disease. Reference level specifically refers to the level of miRNA or miRNA expression quantified in a sample from a healthy subject, from a subject, which is not at risk of or suffering from heartworm disease. Specifically, a more than 1.3, 1.4, 1.5, 1.6, 1.7, 1.8, 1.9 or 2.0 fold difference between the reference level of one or more miRNAs as defined herein obtained from a sample of a subject. Additionally, a control may also be a standard reference value or range of values, i.e. such as stable expressed miRNAs in the samples, for example the endogenous control.

“Animal(s)”, as used herein, unless otherwise indicated, refers to an individual animal that is a mammal. Specifically, mammal refers to a vertebrate animal that is human and non-human, which are members of the taxonomic class Mammalia. Non-exclusive examples of non-human mammals include companion animals. Non-exclusive examples of a companion animal include: dog, cat, and horse, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can further refer to wild animals, including, but not limited to bison, elk, deer, venison, duck, fowl, fish, and the like.

As used herein, the terms “dog” or “canine” are used interchangeably and refer to any member of the Canidae family including, but not limited to, Canis lupus, Canis familiaris, Canis latrans, Canis dingo, Lycaon pictus, Chrysocyon brachyurus, Atelocynus microis, Cuon alpinus, Speothos venaticus, Nyctereutes procyonoides, Vulpes vulpes, and Alopex lagopus. In certain embodiments, the dog or canine is Canis familiaris.

### II. Compositions

The present invention provides genomic identifiers for monitoring heartworm disease. These can be used as target nucleic acid sequences for diagnosis of heartworm disease in a subject.

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well-known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications.

Provided herein is a method for diagnosing heartworm in a subject, comprising the steps of: (a) determining the level of expression of each of a plurality of miRNAs within a sample from a subject; and (b) using one or more Artificial Intelligence (AI) model to diagnose heartworm disease in the subject.

### A. MicroRNAs

MicroRNAs (miRNAs) are small, non-coding RNA molecules involved in the regulation of gene expression. Recent studies have demonstrated that miRNAs are stable in body fluids and their expression profiles can reflect pathological conditions, making them promising biomarkers of diseases, including parasitic infections (Manzano-Román R., Mol. Biochem. Parasitol., 2012). A study conducted in dogs showed that the presence of miR-71 and miR-34 in plasma was able to discriminate heartworm-infected from uninfected animals (Tritten, PLOS Negl. Trop. Dis. 2014), even though it was confirmed that miRNA expression levels did not reflect the intensity of adult worms in the dog (Braman A. A., J. Parasitol., 2018).

Provided herein is a miRNA detection assays that utilizes expression profiling combined with powerful bioinformatic analysis and AI modelling. The development of a miRNA-based diagnostic assay for heartworm infection would offer several advantages over current diagnostic methods. The analysis of the expression level of specific miRNAs (Table 1) would enable early detection of the heartworm infection, including low-burden and male-only infections, which are often missed by antigen tests. Our focus on extracellular miRNAs allows the use of biofluid samples (e.g. blood) in a simple laboratory protocol, in conjunction with bespoke and accurate result modelling. MiRNA-based assays can be more cost-effective and less invasive compared to imaging techniques, providing a reliable and accessible diagnostic tool for veterinarians to diagnose and monitor heartworm infections in dogs.

Nucleotide sequences of mature miRNAs and their respective precursors are known in the art and available from the database miRBase or from Sanger database.

Identical polynucleotides as used herein in the context of a polynucleotide to be detected by the method as described herein may have a nucleic acid sequence with an identity of at least 90%, 95%, 97%, 98% or 99% or less than 3 or 2 single nucleotide modifications compared to a polynucleotide comprising or consisting of the nucleotide sequence of any one of SEQ ID Nos: 1-82.

Furthermore, identical polynucleotides as used herein in the context of a polynucleotide to be detected by the method as described herein may have a nucleic acid sequence with an identity of at least 90%, 95%, 97%, 98% or 99% to a polynucleotide comprising or consisting of the nucleotide sequence of any one SEQ ID NOs: 1-82 including one, two, three or more nucleotides of the corresponding pre-miRNA or cDNA sequence at the 5′end and/or the 3′end of the respective seed sequence.

All of the specified miRNAs used according to the invention also encompass isoforms and variants thereof. For the purpose of the invention, the terms “isoforms and variants” (which have also be termed “isomirs”) of a reference miRNA include trimming variants (5′ trimming variants in which the 5′ dicing site is upstream or downstream from the reference miRNA sequence; 3′ trimming variants: the 3′ dicing site is upstream or downstream from the reference miRNA sequence), or variants having one or more nucleotide modifications (3′ nucleotide addition to the 3′ end of the reference miRNA; nucleotide substitution by changing nucleotides from the miRNA precursor), or the complementary mature microRNA strand including its isoforms and variants (for example for a given 5′ mature microRNA the complementary 3′ mature microRNA and vice-versa). With regard to nucleotide modification, the nucleotides relevant for RNA/RNA binding, i.e. the 5′-seed region and nucleotides at the cleavage/anchor side are excluded from modification.

In the following, if not otherwise stated, the term “miRNA” encompasses 3p and 5p strands and also its isoforms and variants.

The method further comprises the use of at least one normalizer and/or an off-species control miRNA molecule. At least one normalizer is used to ‘normalize’ data, i.e. to control for variation between the samples tested in the method of the invention, and the at least one control is used to try to ensure there are no failure or false readings in the results. An off-species control is added in to show that the miRNAs detected are relevant to the species panel. The off-species control is a miRNA from another species, i.e. not a ruminant. Advantageously, the use of an off-species control provides another layer of control to distinguish between background or non-specific signals and a positive result.

It is preferred that the method comprises the step of assessing the relative levels of miRNA expression of each one of miRNA molecules of SEQ ID NOs: 1-41 within a sample from a subject and using the data obtained from measurement of the expression levels to determine the presence or absence of disease in a subject. The miRNA molecules described herein have a reverse complement cDNA with at least 90% sequence identity to SEQ ID NO: 42-82.

Also provided herein is a kit for use in performing the method of the first aspect comprising means for determining the level of expression of each one of the miRNA molecules of SEQ ID NOs: 1-41, having the reverse compliment cDNA of SEQ ID NOs: 42-82.

B. Dirofilaria immitis (Heartworm)

Provided herein are methods of detecting Dirofilariasis or heartworm disease. Dirofilariasis is a parasitic disease of animals and occasionally in humans, which may result from infection by a species of Dirofilaria such as D. immitis, D. repens, D. tenuis, D. ursi, D. subdermata, D. lutrae, D. striata and D. spectans. Dirofilaria immitis (heartworm) is a parasitic nematode that commonly infects dogs, foxes, wolves, coyotes, and cats. Heartworms may cause serious vascular damage and may be fatal, especially in highly active animals. Heartworm infections in cats, especially those with low worm burdens or only male worms, can be difficult to detect because antigen tests are designed to detect proteins from female worms, and these infections may not produce enough antigen to be detected. A negative antigen test result doesn't always mean a cat is heartworm-free, as infections may be missed due to these factors.

The life cycle of Dirofilaria immitis is well known (reviewed in McCall et al., Adv Parasitol. 66:193-285, 2008). In brief, a mosquito may become infected when it draws blood from an infected host (e.g. a dog). In the mosquito, microfilariae develop to the infective larval stage. When the infected mosquito feeds, it may transmit larvae to a new host (e.g. another dog). In the new host the larvae continue to mature for eight to ten weeks, after which time they move to the right side of the lungs and the pulmonary artery, where they become adult. Adult worms mate and females produce eggs, which develop in utero into the long thin embryos (microfilariae) that are released into the bloodstream. A mosquito that takes in the circulating microfilariae when it draws blood from the infected host starts the cycle again. Dirofilaria immitis may be found wherever its vector, the mosquito, is found. Generally, Dirofilaria immitis may be found on a world-wide basis, but are very common in areas with mild and warm climates. Macrocyclic lactones are often prescribed as therapeutics or prophylactics in the management of Dirofilaria immitis in veterinary applications. However, resistance to macrocyclic lactones is common in a variety of parasitic nematodes and appears to be developing in D. immitis (WO 2011/120165 PCT/CA2011/050169). A number of tests have been described for the detection of anthelmintic resistance in nematodes of livestock and horses, including, fecal egg count reduction test, the egg hatch test, microagar larval development test and molecular tests 5 based on benzimidazole resistance (reviewed in Coles et al., Veterinary Parasitology 136:167-185, 2006). Prichard et al. (European patent EP 0979278) describes a P-glycoprotein sequence in Haemonchus contortus which may be useful for the diagnosis of macrocyclic lactone resistance in parasitic nematodes. However, there remains a need for methods to detect Dirofilaria immitis (heartworms) that are resistant to a macrocyclic lactone.

While Dirofilaria immitis is known to mainly spread to dogs by mosquito bites, causing heart failure in the definitive host canine (Milanez de Campos J. R., et al., Chest. 1997; 112:729-733), other hosts may include cats, wolves, foxes and rarely humans. Dirofilaria immitis is found throughout the world and in the United States is endemic to the east, southeastern seaboard, and the southern coast (Asimacopoulos P. J., et al. Chest. 1992; 102:851-855). In the event that a human is bitten by an infected mosquito, the nematode travels from the subcutaneous tissue into the vessels, and eventually enters the right ventricle.

C. miRNAs for Diagnosing Heartworm Infection or Disease

MicroRNAs (miRNAs) are small, non-coding RNA molecules involved in the regulation of gene expression. Recent studies have demonstrated that miRNAs are stable in body fluids and their expression profiles can reflect pathological conditions, making them promising biomarkers of diseases, including parasitic infections. A study conducted in dogs showed that the presence of miR-71 and miR-34 in plasma was able to discriminate heartworm-infected from uninfected animals (Evans C C, et al. Pathogens. 2022 Sep. 21; 11 (10): 1073), even though it was confirmed that miRNA expression levels did not reflect the intensity of adult worms in the dog.

Provided herein is a miRNA detection assays that utilizes expression profiling combined with powerful bioinformatic analysis and AI modelling. The development of a miRNA-based diagnostic assay for heartworm infection would offer several advantages over current diagnostic methods. The analysis of the expression level of specific miRNAs (Table 1) would enable early detection of the heartworm infection, including low-burden and male-only infections, which are often missed by antigen tests. Our focus on extracellular miRNAs allows the use of biofluid samples (e.g. blood) in a simple laboratory protocol, in conjunction with bespoke and accurate result modelling. MiRNA-based assays can be more cost-effective and less invasive compared to imaging techniques, providing a reliable and accessible diagnostic tool for veterinarians to diagnose and monitor heartworm infections in dogs.

TABLE 1
List of miRNAs and cDNAs utilized to diagnose heartworm
infection in dogs, wherein the asterisk (*) denotes a normalizer
and/or control miRNA molecule.

| | | SEQ | | SEQ |
|---|---|---|---|---|
| miRNA ID | RNA sequence | ID NO | cDNA sequence | ID NO |
| cel-miR-39 | UCACCGGGUGUAAAUCAGCUUG | 1 | TCACCGGGTGTAAATCAGCTTG | 42 |
| let-7a | UGAGGUAGUAGGUUGUAUAGUU | 2 | TGAGGTAGTAGGTTGTATAGTT | 43 |
| let-7b | UGAGGUAGUAGGUUGUGUGGUU | 3 | TGAGGTAGTAGGTTGTGTGGTT | 44 |
| let-7c | UGAGGUAGUAGGUUGUAUGGUU | 4 | TGAGGTAGTAGGTTGTATGGTT | 45 |
| let-7e | UGAGGUAGGAGGUUGUAUAGUU | 5 | TGAGGTAGGAGGTTGTATAGTT | 46 |
| let-7g | UGAGGUAGUAGUUUGUACAGUU | 6 | TGAGGTAGTAGTTTGTACAGTT | 47 |
| let-7i | UGAGGUAGUAGUUUGUGCUGUU | 7 | TGAGGTAGTAGTTTGTGCTGTT | 48 |
| miR-1 | UGGAAUGUAAAGAAGUAUGUA | 8 | TGGAATGTAAAGAAGTATGTA | 49 |
| miR-100d | UACCCGUAGCUCCGAAUAUGUG | 9 | TACCCGTAGCTCCGAATATGTG | 50 |
| miR-10a | UACCCUGUAGAUCCGAAUUUGU | 10 | TACCCTGTAGATCCGAATTTGT | 51 |
| miR-128 | UCACAGUGAACCGGUCUCUUU | 11 | TCACAGTGAACCGGTCTCTTT | 52 |
| miR-130b | CAGUGCAAUGAUGAAAGGGCAU | 12 | CAGTGCAATGATGAAAGGGCAT | 53 |
| miR-133a | UUGGUCCCCUUCAACCAGCUGU | 13 | TTGGTCCCCTTCAACCAGCTGT | 54 |
| miR-133b | UUUGGUCCCCUUCAACCAGCUA | 14 | TTTGGTCCCCTTCAACCAGCTA | 55 |
| miR-142 | CCCAUAAAGUAGAAAGCACUA | 15 | CCCATAAAGTAGAAAGCACTA | 56 |
| miR-155 | UUAAUGCUAAUCGUGAUAGGGGU | 16 | TTAATGCTAATCGTGATAGGGGT | 57 |
| miR-17 | CAAAGUGCUUACAGUGCAGGUAG | 17 | CAAAGTGCTTACAGTGCAGGTAG | 58 |
| miR-206 | UGGAAUGUAAGGAAGUGUGUGG | 18 | TGGAATGTAAGGAAGTGTGTGG | 59 |
| miR-20a | UAAAGUGCUUAUAGUGCAGGUAG | 19 | TAAAGTGCTTATAGTGCAGGTAG | 60 |
| miR-21 | UAGCUUAUCAGACUGAUGUUGA | 20 | TAGCTTATCAGACTGATGTTGA | 61 |
| miR-223 | UGUCAGUUUGUCAAAUACCCC | 21 | TGTCAGTTTGTCAAATACCCC | 62 |
| miR-23a | AUCACAUUGCCAGGGAUUU | 22 | ATCACATTGCCAGGGATTT | 63 |
| miR-26a | UUCAAGUAAUCCAGGAUAGGCU | 23 | TTCAAGTAATCCAGGATAGGCT | 64 |
| miR-29a | UAGCACCAUCUGAAAUCGGUUA | 24 | TAGCACCATCTGAAATCGGTTA | 65 |
| miR-30b | UGUAAACAUCCUACACUCAGCU | 25 | TGTAAACATCCTACACTCAGCT | 66 |
| miR-30c | UGUAAACAUCCUACACUCUCAGCU | 26 | TGTAAACATCCTACACTCTCAGCT | 67 |
| miR-30d | UGUAAACAUCCCCGACUGGAAGCU | 27 | TGTAAACATCCCCGACTGGAAGCT | 68 |
| miR-320 | AAAAGCUGGGUUGAGAGGGCGA | 28 | AAAAGCTGGGTTGAGAGGGCGA | 69 |
| miR-375 | UUUGUUCGUUCGGCUCGCGUGA | 29 | TTTGTTCGTTCGGCTCGCGTGA | 70 |
| miR-423a | UGAGGGGCAGAGAGCGAGACUUU | 30 | TGAGGGGCAGAGAGCGAGACTTT | 71 |
| miR-486 | UCCUGUACUGAGCUGCCCCGAG | 31 | TCCTGTACTGAGCTGCCCCGAG | 72 |
| miR-490* | CAACCUGGAGGACUCCAUGCUG | 32 | CAACCTGGAGGACTCCATGCTG | 73 |
| miR-499 | UUAAGACUUGCAGUGAUGUUU | 33 | TTAAGACTTGCAGTGATGTTT | 74 |
| miR-599 | GUUGUGUCAGUUUAUCAAAC | 34 | GTTGTGTCAGTTTATCAAAC | 75 |
| miR-71 | UGAAAGACAUGGGUAGUGA | 35 | TGAAAGACATGGGTAGTGA | 76 |
| miR-81 | UGAGAUCAUCGUGAAAGCUAGU | 36 | TGAGATCATCGTGAAAGCTAGT | 77 |
| miR-874 | CUGCCCUGGCCCGAGGGACCGA | 37 | CTGCCCTGGCCCGAGGGACCGA | 78 |
| miR-9 | UCUUUGGUUAUCUAGCUGUAUGA | 38 | TCTTTGGTTATCTAGCTGTATGA | 79 |
| miR-92a | UAUUGCACUUGUCCCGGCCUGU | 39 | TATTGCACTTGTCCCGGCCTGT | 80 |
| miR-98 | UGAGGUAGUAAGUUGUAUUGUU | 40 | TGAGGTAGTAAGTTGTATTGTT | 81 |
| miR-99a | AACCCGUAGAUCCGAUCUUGU | 41 | AACCCGTAGATCCGATCTTGT | 82 |

### D. Identification of Target Sequences

The present invention relates to a method for detecting the presence or amount of a target polynucleotide (nucleic acid sequence) from the host's response to heartworm in a sample. The target polynucleotide is a virulence determinant. In a preferred embodiment, the target polynucleotide is miRNA. The invention is also directed to a method of detecting the presence of a disease or infection state in a mammal, by detecting the presence or amount of a target miRNA, wherein the presence or amount of the target miRNA identifies the disease state. Thus, the invention relates to diagnostic compositions and methods for detecting heartworm. The sample containing the target miRNA may be tissue, collection of cells, cell lysate, body fluid, excretum, in vitro culture, purified polynucleotide, isolated polynucleotide, food sample, medical sample, agro-livestock sample, or environmental sample.

In another embodiment, the invention provides a method for capturing, detecting, and quantifying miRNA from its reverse transcribed cDNA. miRNA is extracted from the provided biological sample using commercially available miRNA specific extraction kits and the manufacturer's recommended protocol (e.g. Qiagen miRNeasy Serum/Plasma Kit). From the extracted miRNA, cDNA is reverse transcribed and amplified using commercially available miRNA to cDNA specific extraction kits and the manufacturer's recommended protocol (e.g. TaqMan Advanced miRNA cDNA Synthesis Kit). The resulting reverse transcribed cDNA of the miRNA may be captured and/or detected using the universal sequences added at both the 5′ and 3′ ends and the cDNA product may undergo universal pre-amplification and/or amplification using a single pair of universal forward and reverse primers. The relative expression levels of specific miRNA, which form part of the defined diagnostic panel, are inferred through the relative expression levels of their respective cDNA, i.e. detection by proxy. This can be performed via numerous traditional DNA detection methods, such as qPCR or Next Generation sequencing, or via newer multiplexing techniques such as beads capture technologies such as the Luminex xMAP system.

The invention described here utilizes large-scale identification of disrupted genes and the use of bioinformatics and AI to select mutants that could be characterized in animals.

E. Multiplex miRNA Profiling

The present invention uses multiplex miRNA profiling without RNA purification. Accuracy of miRNA profiling is enhanced when sample processing can be kept to a minimum, avoiding steps such as RNA purification that can introduce bias and inaccuracies. The present invention used a multiplex circulating miRNA assay that enables the profiling of a plurality of miRNAs in the same well directly from the sample, with no need for RNA purification. In one embodiment, the assay uses Luminex xMAP system beads, which enable the multiplex capture of miRNAs with picomolar sensitivity and high specificity. The Luminex xMAP beads functions with bespoke probes which contain three distinct functional regions: a complementary DNA section to the relevant DNA tag on the Luminex xMAP bead; an RNA region complimentary to the target miRNA; and a biotin fluorescent reporter tag. Detection is carried out using a Luminex LX200, or other compatible device, to detect miRNA molecules that emit fluorescence that is proportional to their abundance in the sample. Each miRNA that was used was given a unique bead region (up to 80 different regions were possible). The data that was obtained from the mixture of particles could then be attributed to the miRNAs by identification of the code.

The present invention uses multiplex miRNA relative expression profiling with machine learning and predictive classification analysis. Accuracy of miRNA expression profiling is enhanced when marker expression is analyzed relative to all other markers, as this allows detection of both increased and decreased expression, something not possible with simple threshold level analysis. The present invention used a RT-qPCR approach, with an option step of detection form synthesized cDNA to enhance sensitivity of detection. In one embodiment, the assay uses sample RNA extraction (e.g. Qigen miRNeasy kits), cDNA synthesis and amplification (e.g. TaqMan™ Advanced miRNA cDNA Synthesis Kit) followed by RT-qPCR detection with marker specific primers (e.g. TaqMan™ Advanced miRNA assays). Detection is carried out using a QuantStudio 5 Real-Time PCR Systems, or other RT-qPCR compatible device, to detect miRNA molecules that emit fluorescence that is proportional to their abundance in the samples. An alternate embodiment may utilize Luminex xMAP system beads, which enable the multiplex capture of miRNAs with picomolar sensitivity and high specificity. The Luminex xMAP beads function with bespoke probes which contain three distinct functional regions: a complementary DNA section to the relevant DNA tag on the Luminex xMAP bead; an RNA region complimentary to the target miRNA; and a biotin fluorescent reporter tag. Detection is carried out using a Luminex LX200, or other compatible device, to detect miRNA molecules that emit fluorescence that is proportional to their abundance in the sample. Each miRNA that was used was given a unique bead region (up to 80 different regions were possible). The data that was obtained from the mixture of particles could then be attributed to the miRNAs by identification of the code.

The disease is selected from the group consisting of heartworm and related conditions.

The sample or blood sample refers to tissue or organ sample, blood, cell-free blood such as serum and plasma, urine, saliva, milk and cerebrospinal fluid sample.

From the results of the experiments below, a differentiation in expression levels of miRNA was identified when comparing healthy animals with animals that have heartworm.

### F. Predictive Modeling

Provided herein are methods using predictive modelling to investigate the scope to use the miRNA profiles to predict the presence or absence of disease. A group of healthy and unhealthy animals were taken and tested to determine the level of miRNA expression in samples from these animals. The data obtained was then used to train the models.

Fifteen machine learning models were fitted and compared with the aim of obtaining the best predictions of the disease outcome. Formal assessment of performance was conducted by computing a number of performance statistics based on 5-time repeated 10-fold cross-validation. Cross-validation was useful to obtain more realistic model performance measures from the training data.

Data from the RT-qPCR analysis from each of the miRNA molecules from Table 1 was fitted to each of the models.

This disclosed invention utilized microRNA (miRNA) expression profile analysis to distinguish heartworm cases from healthy controls in canine serum samples. A proprietary bioinformatic pipeline incorporating machine learning was used to optimize predictive classification models, achieving an overall accuracy of 70%, sensitivity: 0.77, specificity: 0.6, ROC AUC: 0.88. Additionally, principal component analysis (PCA) and clustering confirmed that heartworm-associated miRNA profiles were distinct from controls but similar between urine and serum, promoting urine as an additional sample medium. The disclosed methods successfully demonstrated that miRNA biomarkers can differentiate heartworm from healthy cases and are detectable in urine.

For any method disclosed herein that includes discrete steps, the steps may be conducted in any feasible order. And, as appropriate, any combination of two or more steps may be conducted simultaneously.

The description exemplifies illustrative embodiments. In several places throughout the application, guidance is provided through lists of examples, which examples can be used in various combinations. In each instance, the recited list serves only as a representative group and should not be interpreted as an exclusive list.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### EXAMPLES

The present invention is illustrated by the following examples. It is to be understood that the particular examples, materials, amounts, and procedures are to be interpreted broadly in accordance with the scope and spirit of the invention as set forth herein.

### Example: Testing of qPCR for miRNA Profiling: Canine Heartworm

### Materials and Methods

In total, 76 samples (39 negative controls and 37 microscopy-positive heartworm cases) were reverse transcribed and assayed using TaqMan Advanced miRNA cDNA Synthesis kits specific to each miRNA. miRNAs with 20%>missing data or where the duplicates Ct values were more than 2 SD were removed. Standard deviation and CoV were used to identify a marker with low variability and high amplification suitable for normalisation (mir490). Data for the remaining host miRNAs with 80% amplification rates were then normalised using delta Ct against mir490. Parasite-specific miRNA RT-qPCR was performed in a subset of samples (n=48), showing that the parasite-specific miRNA, mir71, had >80% amplification rate in these data. Variable importance was assessed for each individual host miRNA using a filter-based ROC analysis, suggesting that three had some potential to classify positive and negative samples (AUC>0.6; mir223, mir20a, mir375). A GLM was built using the expression profile of these host miRNAs and its classification metrics investigated. A GLM using the subset of sample assayed using mir71 RT-qPCR was also fitted and tested for classification (n=37).

### Results

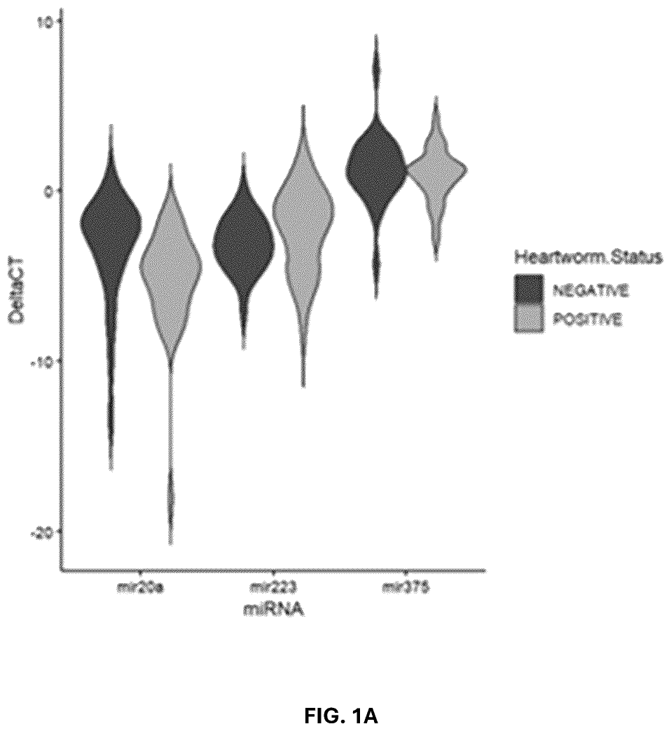

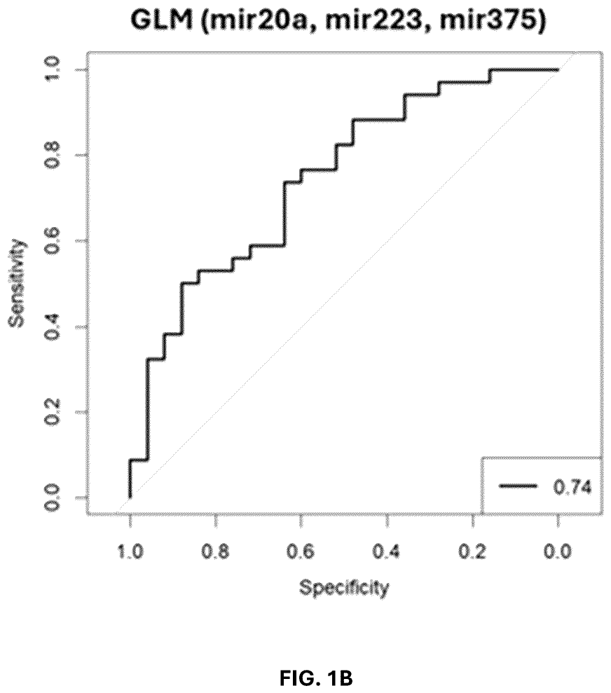

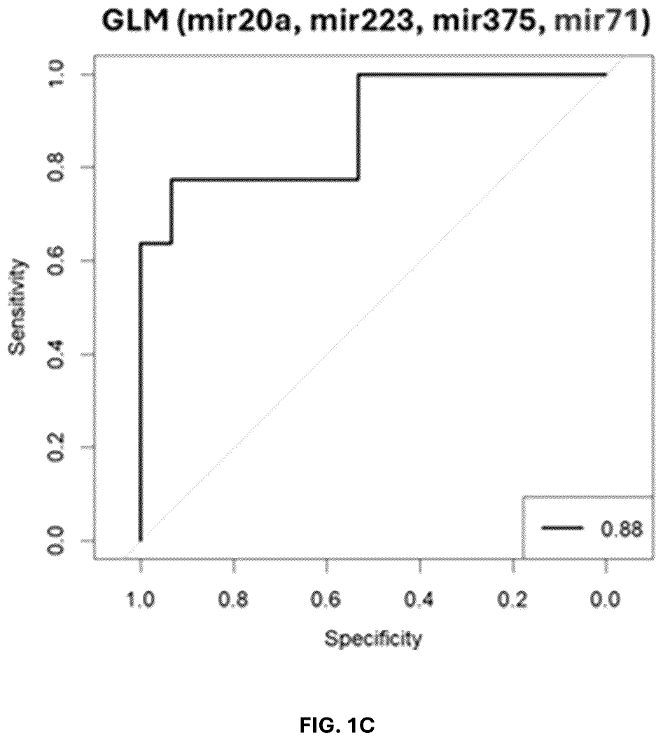

After filtering for samples with missing data for mir223, mir20a, and mir375, 39 positive and 30 negative samples remained (n=59). A violin plot suggested some separation of expression levels for these miRNAs (FIG. 1A). There was a degree of overlap between expression distributions, suggesting similar profiles for some samples across the negative and positive groups, indicating possible heterogeneity in the population. Despite this, the overall accuracy of the fitted GLM was 68%, sensitivity 0.79, specificity 0.52, and ROC AUC 0.74 (FIG. 1B). A GLM fitted to the subset of data probed with a parasite-specific miRNA qPCR (n=37) showed that the addition of mir71 to the GLM increased classification metrics (accuracy 70%, sensitivity: 0.77, specificity: 0.6, ROC AUC: 0.88 (FIG. 1C). While mir223 alone showed specificity, the addition of other markers showed an increase in specificity. Each marker showed specificity for its own a subset of samples. Mir423a for example, was shown to be useful for a subset of samples but isn't quite as important for the wider data set as there was a lot of sub-structure. These results leading to the conclusion that a miRNA panel for diagnosis, would likely have more than one miRNA, which would be useful for providing increased specificity of a diagnostic panel.

These results suggest RT-qPCR miRNA profiling using the TaqMan Advanced miRNA cDNA Synthesis system can diagnose canine heartworm in the training data using a GLM, despite a large overlap in miRNA expression profiles between negative and positive groups. This overlap suggests heterogeneity in the positive population that may indicate asymptomatic, early infection or differential drug responses. Stratification of positive cases is supported by the literature, but further metadata will be required to fully investigate this hypothesis. The addition of a parasite-specific miRNA (mir71) in a subset of samples increased classification metrics within the reduced dataset.

What is claimed is:

1. A method for diagnosing heartworm infection in a subject, comprising the steps of:
a. obtaining a sample from the subject;
b. isolating miRNA molecules within a sample from a subject;
c. amplifying the cDNA molecules to a detectable concentration;
d. probing for the cDNA molecules complementary to the desired miRNA markers;
e. determining a level of expression of the miRNA molecules within a sample from a subject by the level of cDNA molecules probed for the desired miRNA markers; and
f. using one or more Artificial Intelligence (AI) model to predict the disease condition of the subject;
wherein the one or more AI model compares the level of expression of each cDNA molecule with at least one pre-determined reference level cDNA molecule characteristic of a non-diseased subject wherein a deviation of the level of expression of said cDNA molecule in comparison with the at least one reference level cDNA molecule allows for the diagnosis and/or prognosis of heartworm disease, and wherein the miRNA molecules comprise a panel of reference miRNA molecules selected from miRNA molecules having at least one miRNA selected from a group consisting of nucleic acid sequence having at least 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, 41, or combinations thereof, the miRNA molecules having a reverse complement cDNA with at least 99% sequence identity to SEQ ID NO: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 or combinations thereof.

2. The method according to claim 1, wherein the method further comprises the step of using a machine learning algorithm for predictive modelling.

3. The method according to claim 1, wherein the cDNA molecule may also be a reverse complement cDNA.

4. The method according to claim 1, wherein the panel of miRNA molecules comprises at least six reference miRNA molecules, wherein the at least six miRNA molecules are mir20a, mir223a, mir375, mir29a, mir423a, and mir71, having at least 99% sequence identity to SEQ ID NO: 19, 21, 29, 24, 30, and 35, respectively, the miRNA molecules having a reverse complement cDNA with at least 99% sequence identity to SEQ ID NO: 60, 62, 70, 62, 71, and 76, respectively.

5. The method according to claim 1, wherein the panel of miRNA molecules comprises at least five reference miRNA molecules, wherein the at least five miRNA molecules are mir20a, mir223a, mir375, mir29a, and mir423a, having at least 99% sequence identity to SEQ ID NO: 19, 21, 29, 24, and 30, respectively, the miRNA molecules having a reverse complement cDNA with at least 99% sequence identity to SEQ ID NO: 60, 62, 70, 62, and 71, respectively.

6. The method according to claim 1, wherein the panel of miRNA molecules comprises at least four reference miRNA molecules, wherein the at least four miRNA molecules are mir20a, mir223a, mir375, and mir71, having at least 99% sequence identity to SEQ ID NO:19, 21, 29, and 35, respectively, the miRNA molecules having a reverse complement cDNA with at least 99% sequence identity to SEQ ID NO: 60, 62, 70, and 76, respectively.

7. The method according to claim 1, wherein the panel of miRNA molecules comprises at least three reference miRNA molecules, wherein the at least three miRNA molecules are mir20a, mir223a, and mir375, having at least 99% sequence identity to SEQ ID NO: 19, 21, and 29, respectively, the miRNA molecules having a reverse complement cDNA with at least 99% sequence identity to SEQ ID NO: 60, 62, and 70, respectively.

8. The method according to claim 1, wherein the panel of miRNA molecules comprises at least two reference miRNA molecules, wherein the at least two miRNA molecules are mir223 and mir71, having at least 99% sequence identity to SEQ ID NO: 21 and 35, the miRNA molecules having a reverse complement cDNA with at least 99% sequence identity to SEQ ID NO:62 and 76.

9. The method according to claim 1, wherein the panel of miRNA molecules comprises at least one reference miRNA molecule, wherein the at least one miRNA molecule is mir223, having at least 99% sequence identity to SEQ ID NO:21, the miRNA molecules having a reverse complement cDNA with at least 99% sequence identity to SEQ ID NO:62.

10. The method according to claim 1, wherein the method further comprises the use of a normalizer and/or control miRNA molecule, wherein the normalizer miRNA molecule is mir490, having at least 99% sequence identity to SEQ ID NO:32, the miRNA molecules having a reverse complement cDNA with at least 99% sequence identity to SEQ ID NO:73.

11. The method according to claim 10, wherein the normalizer or control miRNA molecule is an off-species control miRNA molecule.

12. The method according to claim 1, wherein the miRNAs are cell free miRNAs.

13. The method according to claim 1, wherein the subject is a mammal.

14. The method according to claim 1, wherein the subject is a dog, cat, or horse.

15. The method according to claim 1, wherein the sample is selected from a group consisting of a tissue or organ sample, blood samples, urine, saliva, milk, and cerebrospinal fluid sample.

16. The method according to claim 15, wherein the blood sample is selected from the group consisting of serum, plasma, cell-free blood, whole blood, and its components, blood-derived products, or preparations thereof.

17. A kit for use in performing the method of claim 1 comprising means for determining the level of expression of miRNA molecules selected from a miRNA panel having one or more miRNA molecules selected from miRNA molecules having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, 41, or combinations thereof, the miRNA molecules having a reverse complement cDNA with at least 99% sequence identity to SEQ ID NO: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or combinations thereof.

18. A method of selecting a panel for use in heartworm diagnosis comprising the steps of:
(a) selecting a group of miRNA molecules or the reverse complement cDNA according to claim 1, the differential expression of which may be associated with a disease condition;
(b) predicting the disease condition based on a deviation of the level of expression of said miRNA molecules from steps (a) and (b); and
(c) reducing the number of miRNAs in the panel to a minimum number to provide a panel of miRNAs that still produces a result.


SEQUENCE LISTING (82 sequences)

SEQ ID NO: 1 - RNA, 22 nt, synthetic construct
  source 1..22: mol_type = other RNA
tcaccgggtgtaaatcagcttg

SEQ ID NO: 2 - RNA, 22 nt, synthetic construct
  source 1..22: mol_type = other RNA
tgaggtagtaggttgtatagtt

SEQ ID NO: 3 - RNA, 22 nt, synthetic construct
  source 1..22: mol_type = other RNA
tgaggtagtaggttgtgtggtt

SEQ ID NO: 4 - RNA, 22 nt, synthetic construct
  source 1..22: mol_type = other RNA
tgaggtagtaggttgtatggtt

SEQ ID NO: 5 - RNA, 22 nt, synthetic construct
  source 1..22: mol_type = other RNA
tgaggtaggaggttgtatagtt

SEQ ID NO: 6 - RNA, 22 nt, synthetic construct
  source 1..22: mol_type = other RNA
tgaggtagtagtttgtacagtt

SEQ ID NO: 7 - RNA, 22 nt, synthetic construct
  source 1..22: mol_type = other RNA
tgaggtagtagtttgtgctgtt

SEQ ID NO: 8 - RNA, 21 nt, synthetic construct
  source 1..21: mol_type = other RNA
tggaatgtaaagaagtatgta

SEQ ID NO: 9 - RNA, 22 nt, synthetic construct
  source 1..22: mol_type = other RNA
tacccgtagctccgaatatgtg

SEQ ID NO: 10 - RNA, 22 nt, synthetic construct
  source 1..22: mol_type = other RNA
taccctgtagatccgaatttgt

SEQ ID NO: 11 - RNA, 21 nt, synthetic construct
  source 1..21: mol_type = other RNA
tcacagtgaaccggtctcttt

SEQ ID NO: 12 - RNA, 22 nt, synthetic construct
  source 1..22: mol_type = other RNA
cagtgcaatgatgaaagggcat

SEQ ID NO: 13 - RNA, 22 nt, synthetic construct
  source 1..22: mol_type = other RNA
ttggtccccttcaaccagctgt

SEQ ID NO: 14 - RNA, 22 nt, synthetic construct
  source 1..22: mol_type = other RNA
tttggtccccttcaaccagcta

SEQ ID NO: 15 - RNA, 21 nt, synthetic construct
  source 1..21: mol_type = other RNA
cccataaagtagaaagcacta

SEQ ID NO: 16 - RNA, 23 nt, synthetic construct
  source 1..23: mol_type = other RNA
ttaatgctaatcgtgataggggt

SEQ ID NO: 17 - RNA, 23 nt, synthetic construct
  source 1..23: mol_type = other RNA
caaagtgcttacagtgcaggtag

SEQ ID NO: 18 - RNA, 22 nt, synthetic construct
  source 1..22: mol_type = other RNA
tggaatgtaaggaagtgtgtgg

SEQ ID NO: 19 - RNA, 23 nt, synthetic construct
  source 1..23: mol_type = other RNA
taaagtgcttatagtgcaggtag

SEQ ID NO: 20 - RNA, 22 nt, synthetic construct
  source 1..22: mol_type = other RNA
tagcttatcagactgatgttga

SEQ ID NO: 21 - RNA, 21 nt, synthetic construct
  source 1..21: mol_type = other RNA
tgtcagtttgtcaaatacccc

SEQ ID NO: 22 - RNA, 19 nt, synthetic construct
  source 1..19: mol_type = other RNA
atcacattgccagggattt

SEQ ID NO: 23 - RNA, 22 nt, synthetic construct
  source 1..22: mol_type = other RNA
ttcaagtaatccaggataggct

SEQ ID NO: 24 - RNA, 22 nt, synthetic construct
  source 1..22: mol_type = other RNA
tagcaccatctgaaatcggtta

SEQ ID NO: 25 - RNA, 22 nt, synthetic construct
  source 1..22: mol_type = other RNA
tgtaaacatcctacactcagct

SEQ ID NO: 26 - RNA, 24 nt, synthetic construct
  source 1..24: mol_type = other RNA
tgtaaacatcctacactctcagct

SEQ ID NO: 27 - RNA, 24 nt, synthetic construct
  source 1..24: mol_type = other RNA
tgtaaacatccccgactggaagct

SEQ ID NO: 28 - RNA, 22 nt, synthetic construct
  source 1..22: mol_type = other RNA
aaaagctgggttgagagggcga

SEQ ID NO: 29 - RNA, 22 nt, synthetic construct
  source 1..22: mol_type = other RNA
tttgttcgttcggctcgcgtga

SEQ ID NO: 30 - RNA, 23 nt, synthetic construct
  source 1..23: mol_type = other RNA
tgaggggcagagagcgagacttt

SEQ ID NO: 31 - RNA, 22 nt, synthetic construct
  source 1..22: mol_type = other RNA
tcctgtactgagctgccccgag

SEQ ID NO: 32 - RNA, 22 nt, synthetic construct
  source 1..22: mol_type = other RNA
caacctggaggactccatgctg

SEQ ID NO: 33 - RNA, 21 nt, synthetic construct
  source 1..21: mol_type = other RNA
ttaagacttgcagtgatgttt

SEQ ID NO: 34 - RNA, 20 nt, synthetic construct
  source 1..20: mol_type = other RNA
gttgtgtcagtttatcaaac

SEQ ID NO: 35 - RNA, 19 nt, synthetic construct
  source 1..19: mol_type = other RNA
tgaaagacatgggtagtga

SEQ ID NO: 36 - RNA, 22 nt, synthetic construct
  source 1..22: mol_type = other RNA
tgagatcatcgtgaaagctagt

SEQ ID NO: 37 - RNA, 22 nt, synthetic construct
  source 1..22: mol_type = other RNA
ctgccctggcccgagggaccga

SEQ ID NO: 38 - RNA, 23 nt, synthetic construct
  source 1..23: mol_type = other RNA
tctttggttatctagctgtatga

SEQ ID NO: 39 - RNA, 22 nt, synthetic construct
  source 1..22: mol_type = other RNA
tattgcacttgtcccggcctgt

SEQ ID NO: 40 - RNA, 22 nt, synthetic construct
  source 1..22: mol_type = other RNA
tgaggtagtaagttgtattgtt

SEQ ID NO: 41 - RNA, 21 nt, synthetic construct
  source 1..21: mol_type = other RNA
aacccgtagatccgatcttgt

SEQ ID NO: 42 - DNA, 22 bp, synthetic construct
  source 1..22: mol_type = other DNA
tcaccgggtgtaaatcagcttg

SEQ ID NO: 43 - DNA, 22 bp, synthetic construct
  source 1..22: mol_type = other DNA
tgaggtagtaggttgtatagtt

SEQ ID NO: 44 - DNA, 22 bp, synthetic construct
  source 1..22: mol_type = other DNA
tgaggtagtaggttgtgtggtt

SEQ ID NO: 45 - DNA, 22 bp, synthetic construct
  source 1..22: mol_type = other DNA
tgaggtagtaggttgtatggtt

SEQ ID NO: 46 - DNA, 22 bp, synthetic construct
  source 1..22: mol_type = other DNA
tgaggtaggaggttgtatagtt

SEQ ID NO: 47 - DNA, 22 bp, synthetic construct
  source 1..22: mol_type = other DNA
tgaggtagtagtttgtacagtt

SEQ ID NO: 48 - DNA, 22 bp, synthetic construct
  source 1..22: mol_type = other DNA
tgaggtagtagtttgtgctgtt

SEQ ID NO: 49 - DNA, 21 bp, synthetic construct
  source 1..21: mol_type = other DNA
tggaatgtaaagaagtatgta

SEQ ID NO: 50 - DNA, 22 bp, synthetic construct
  source 1..22: mol_type = other DNA
tacccgtagctccgaatatgtg

SEQ ID NO: 51 - DNA, 22 bp, synthetic construct
  source 1..22: mol_type = other DNA
taccctgtagatccgaatttgt

SEQ ID NO: 52 - DNA, 21 bp, synthetic construct
  source 1..21: mol_type = other DNA
tcacagtgaaccggtctcttt

SEQ ID NO: 53 - DNA, 22 bp, synthetic construct
  source 1..22: mol_type = other DNA
cagtgcaatgatgaaagggcat

SEQ ID NO: 54 - DNA, 22 bp, synthetic construct
  source 1..22: mol_type = other DNA
ttggtccccttcaaccagctgt

SEQ ID NO: 55 - DNA, 22 bp, synthetic construct
  source 1..22: mol_type = other DNA
tttggtccccttcaaccagcta

SEQ ID NO: 56 - DNA, 21 bp, synthetic construct
  source 1..21: mol_type = other DNA
cccataaagtagaaagcacta

SEQ ID NO: 57 - DNA, 23 bp, synthetic construct
  source 1..23: mol_type = other DNA
ttaatgctaatcgtgataggggt

SEQ ID NO: 58 - DNA, 23 bp, synthetic construct
  source 1..23: mol_type = other DNA
caaagtgcttacagtgcaggtag

SEQ ID NO: 59 - DNA, 22 bp, synthetic construct
  source 1..22: mol_type = other DNA
tggaatgtaaggaagtgtgtgg

SEQ ID NO: 60 - DNA, 23 bp, synthetic construct
  source 1..23: mol_type = other DNA
taaagtgcttatagtgcaggtag

SEQ ID NO: 61 - DNA, 22 bp, synthetic construct
  source 1..22: mol_type = other DNA
tagcttatcagactgatgttga

SEQ ID NO: 62 - DNA, 21 bp, synthetic construct
  source 1..21: mol_type = other DNA
tgtcagtttgtcaaatacccc

SEQ ID NO: 63 - DNA, 19 bp, synthetic construct
  source 1..19: mol_type = other DNA
atcacattgccagggattt

SEQ ID NO: 64 - DNA, 22 bp, synthetic construct
  source 1..22: mol_type = other DNA
ttcaagtaatccaggataggct

SEQ ID NO: 65 - DNA, 22 bp, synthetic construct
  source 1..22: mol_type = other DNA
tagcaccatctgaaatcggtta

SEQ ID NO: 66 - DNA, 22 bp, synthetic construct
  source 1..22: mol_type = other DNA
tgtaaacatcctacactcagct

SEQ ID NO: 67 - DNA, 24 bp, synthetic construct
  source 1..24: mol_type = other DNA
tgtaaacatcctacactctcagct

SEQ ID NO: 68 - DNA, 24 bp, synthetic construct
  source 1..24: mol_type = other DNA
tgtaaacatccccgactggaagct

SEQ ID NO: 69 - DNA, 22 bp, synthetic construct
  source 1..22: mol_type = other DNA
aaaagctgggttgagagggcga

SEQ ID NO: 70 - DNA, 22 bp, synthetic construct
  source 1..22: mol_type = other DNA
tttgttcgttcggctcgcgtga

SEQ ID NO: 71 - DNA, 23 bp, synthetic construct
  source 1..23: mol_type = other DNA
tgaggggcagagagcgagacttt

SEQ ID NO: 72 - DNA, 22 bp, synthetic construct
  source 1..22: mol_type = other DNA
tcctgtactgagctgccccgag

SEQ ID NO: 73 - DNA, 22 bp, synthetic construct
  source 1..22: mol_type = other DNA
caacctggaggactccatgctg

SEQ ID NO: 74 - DNA, 21 bp, synthetic construct
  source 1..21: mol_type = other DNA
ttaagacttgcagtgatgttt

SEQ ID NO: 75 - DNA, 20 bp, synthetic construct
  source 1..20: mol_type = other DNA
gttgtgtcagtttatcaaac

SEQ ID NO: 76 - DNA, 19 bp, synthetic construct
  source 1..19: mol_type = other DNA
tgaaagacatgggtagtga

SEQ ID NO: 77 - DNA, 22 bp, synthetic construct
  source 1..22: mol_type = other DNA
tgagatcatcgtgaaagctagt

SEQ ID NO: 78 - DNA, 22 bp, synthetic construct
  source 1..22: mol_type = other DNA
ctgccctggcccgagggaccga

SEQ ID NO: 79 - DNA, 23 bp, synthetic construct
  source 1..23: mol_type = other DNA
tctttggttatctagctgtatga

SEQ ID NO: 80 - DNA, 22 bp, synthetic construct
  source 1..22: mol_type = other DNA
tattgcacttgtcccggcctgt

SEQ ID NO: 81 - DNA, 22 bp, synthetic construct
  source 1..22: mol_type = other DNA
tgaggtagtaagttgtattgtt

SEQ ID NO: 82 - DNA, 21 bp, synthetic construct
  source 1..21: mol_type = other DNA
aacccgtagatccgatcttgt